(19) United States
(12) Patent Application Publication
(10) Pub. No.: US 2021/0289716 A1
(43) Pub. Date: Sep. 23, 2021

(54) LIGHT BOX FOR MAINTAINING BIOLOGICAL MATTER

(71) Applicant: University of Tennessee Research Foundation, Knoxville, TN (US)

(72) Inventor: Ansul Lokdarshi, Knoxville, TN (US)

(21) Appl. No.: 17/075,445

(22) Filed: Oct. 20, 2020

Related U.S. Application Data
(60) Provisional application No. 62/990,781, filed on Mar. 17, 2020.

(51) Int. Cl. A01G 9/24; A01N 3/00; C12M 1/00; A01G 7/04

(52) U.S. Cl. CPC A01G 9/246; A01N 3/00; C12M 21/02; A01G 7/045; C12M 41/14; C12M 29/20; A01G 9/249; C12M 31/10

(57) ABSTRACT

An enclosure for maintaining biological matter therein under controlled conditions can include a main body, which has an interior space and a door through which the interior space is accessible, one or more light emitter panels that illuminate the interior space, a base that receives the main body thereon and comprises a cooling system. The cooling system can have a container, a fluid inlet for introducing a fluid into the container, and a fluid outlet for removing the fluid from the container; and a top section that is removably attached to a top surface of the main body. Such an enclosure is capable of maintaining a temperature within the interior space between about 14° C. and about 30° C.

### CROSS-REFERENCE TO RELATED APPLICATIONS

This application claims priority to U.S. Provisional Patent Application Ser. No. 62/990,781, filed Mar. 17, 2020, the disclosure of which is incorporated herein by reference in its entirety.

### STATEMENT OF GOVERNMENT INTEREST

This invention was made with Government support under Contract No. MCB-1546402 awarded by the National Science Foundation. The Government has certain rights in the invention.

### BACKGROUND

Evaluating the optimal growth conditions for biological matter (e.g., plants, unicellular organisms, and the like) has long proven difficult due to the inherent difficulties in producing a sufficiently closed environment in order to properly isolate variables that may impact growth and/or reproduction of the biological matter of interest. As such, a need exists for an enclosure (e.g., a “light box”) that is able to control the light color, the light intensity, the air flow rate and direction, the temperature, and/or the composition of the air within the enclosure. Such an enclosure will allow for controlled experimentation regarding how specific environmental variables influence the growth of biological matter therein.

### SUMMARY

The subject matter disclosed herein provides various aspects of a novel light box for biological matter (e.g., plants, microorganisms (e.g., microalgae), cyanobacteria, bacteria, and yeast cultures and the like) under controlled environmental conditions. More particularly, the light box can provide a small, portable, illuminated growth chamber for photosynthetic organisms (e.g., plants and cyanobacteria) and can be modified for bacteria and yeast culture. In some embodiments, the light box can be used for studying the growth of plant research models, such as Arabidopsis thaliana, Medicago truncatula, Nicotiana benthamiana, among others, and microgreens such as lettuce, basil, among others. The presently disclosed light box can maintain physiological temperatures (e.g., an interior temperature of between about 14° C. and about 30° C.) via the use of a bottom mounted cooling system. In some embodiments, the light box can also include a fan, e.g., mounted near the top of the light box or near one or more light sources inside the light box, to facilitate air circulation and cooling. The fan can be regulated based upon the light intensity inside the light box and/or the interior temperature. The combination of the bottom mounted cooling system and top-mounted fan can provide improved/more efficient cooling compared to light boxes comprising a top-mounted cooling system. In some embodiments, the combination of air inlet, exhaust vent, and exhaust fan placement can provide for maintenance of the interior temperature in a desired temperature range, while at the same time, maintaining reduced noise levels from fan operation.

Thus, in some embodiments, the presently disclosed light box can comprise (a) a main body or structure comprising or defining an accessible interior space, with one or more light sources (e.g., light bulbs, LEDs, etc.) configured to light the accessible interior space; and (b) a base, wherein the main body is configured to fit on the base and wherein the base comprises a cooling system. In some embodiments, the main body is substantially cuboid-shaped. In some embodiments, the main body comprises a top side, a bottom side, a front side, a rear (or back) side, a left side, and a right side that define the interior space. In some embodiments, the interior space is accessible via at least one door in one of the sides. Additionally or alternatively, in some embodiments, the interior of the light box can be accessible via removal of the top side of the structure. Thus, in some embodiments, the top side can comprise a removable lid or cover. In some embodiments, for example, to cater to the type of sample being grown in the light box (e.g., plant seedlings on plates versus plants in pots with soil), access to the interior of the chamber can be both from the top (e.g., by way of a removable lid or cover) and from one or more side of the chamber.

In some embodiments, one of the sides is configured to hold the fan. In some embodiments, the top side is configured to hold the fan. In some embodiments, the bottom side of the main body can comprise one or more holes (e.g., evenly spaced on the bottom side) to facilitate cooling of the interior space of the main body via air flow from the base comprising the cooling system.

In some embodiments, the cooling system can comprise a container (e.g., an aluminum or other metal container), through which a cooled liquid can be flowed. The cooling system or container can include an inlet for introducing a cooled liquid into the container and an outlet for removing the cooled liquid from the container. In some embodiments, the container is a metal coil (e.g., an aluminum coil) or a metal cylinder (e.g., an aluminum cylinder), optionally containing one or more projections designed to increase the surface area of the container. In some embodiments, the metal coil or the metal cylinder is made of stainless steel and/or Heresite P413 or E-coated aluminum and/or copper nickel alloy. In some embodiments, the cooling system further comprises a reservoir, e.g. holding water or a water/ice mixture, and a pump configured to pump cooled liquid from the reservoir into the container. Alternatively, the inlet can be configured to attach to a cold liquid source, e.g., a cold water faucet, and the outlet can be configured for attachment to a hose leading to liquid recovery tank or a drain. In some embodiments, the light box can include a thermoelectric cooling system, e.g., instead of or in addition to the cooling system through which a cooled liquid can be flowed (e.g., an evaporative cooling system). In some embodiments, the light box includes two cooling systems, an evaporative cooling system and a thermoelectric cooling system.

In some embodiments, the light box can comprise one or more optional air inlet vents (e.g., on a front, back, left, or right side of main body) configured to allow air inflow.

The presently disclosed light box can have a smaller size and lower weight compared to currently commercially available culture/incubation chambers, while still providing space for the inclusion of replicate samples. For example, in some embodiments, the presently disclosed light box can be small enough to fit into the trunk of a car. In some embodiments, the light box can have a length of about 19 inches, a width of about 14 to about 15 inches (e.g., about 14 5/32 inches), and a height of about 18 to 19 inches (including the exhaust fan-containing lid). The main body of the light box can also be prepared from light-weight materials, such as plastics (e.g., acrylics), to further improve ease of mobility and handling. Thus, in some embodiments, the light box can weigh less than about 50 pounds, less than about 45 pounds, less than about 40 pounds, less than about 35 pounds, less than about 30 pounds, or less than about 25 pounds. In some embodiments, the light box can weigh about 25 pounds or about 20 pounds. Due to its small size, portability, and low costs (e.g., less than one thousand dollars or less than 500 dollars), the presently disclosed light box is particularly useful in educational applications (e.g., in K-12 science classes) and/or for field work. In addition, the light box can be powered via battery (e.g., a 12 or 24 Volt battery) in circumstances where relocating plants and/or microorganisms to different places is likely.

The exterior color of the light box can be any color. In some embodiments, the exterior color can be white (e.g., for general use) or can be painted with a color or type of paint that can block external light from the interior of the box, thereby eliminating the effects of external light on sample growth. In some embodiments, the exterior color is black. In some embodiments, the interior space is white, e.g., for high light reflection.

In some embodiments, the light used to illuminate the interior of the light box can be from light emitting diodes (LEDs). In some embodiments, the intensity of the lights can be adjusted. In some embodiments, the lights can be positioned at the top of the interior space and/or near the fan. In some embodiments, the light box can be equipped with separate LED panels for bright white light (>6000 lumens), far red light (e.g., light having a wavelength between about 700 nm and about 780 nm), and blue light (e.g., light having a wavelength between about 400 nm and about 450 nm or between about 400 nm and about 495 nm). In some embodiments, only one type (e.g., white or far-red or blue light) or any combination of light (e.g., far-red with blue) can be used to illuminate the interior of the box. Thus, the light box can be used for studies related to the effects of light intensity/quality on plant growth under different types of abiotic and biotic stresses. Furthermore, the use of LEDs can improve energy and cost management, while the use of LED panels can facilitate vertical and/or horizontal adjustment of the placement of the LED lights within the interior of the light box and interchangeability of position of different panels. For example, the interior of the light box can be equipped with mounts or brackets having slots for the placement of the LED panels at different heights inside the interior space and/or at different positions along one of the front, rear, left, or right sides. Thus, in some embodiments, the mounts can provide for adjustment of LED panel placement along a length or width of the interior space. This versatility can provide for use of the light box with plants of different sizes/ages.

Conditions within and outside of the light box can be monitored, e.g., in real time, via one or more sensors, e.g., humidity, oxygen, carbon dioxide, temperature, light intensity, soil moisture, etc. In some embodiments, the one or more sensors can be integrated and/or can provide data to a display mounted on an outer/exterior surface of the light box. In some embodiments, the one or more sensors can be mounted in an interior of the light box for detecting and/or monitoring environmental conditions within the light box. In some embodiments, the one or more sensors include a sensor mounted on an exterior of the light box for detecting and/or monitoring environmental conditions external to the light box. In some embodiments, a sensor may be provided at an air inlet vent to detect environmental parameters of an air stream drawn into the light box from the exterior of the light box. In some embodiments, the data from one or more of the sensors can be monitored remotely, e.g., via a wireless transmitter/receiver. Thus, in some embodiment, the light box further comprises a microprocessor (e.g., a Raspberry Pi) configured for communication with the one or more sensors and to display one or more conditions within the interior space of the light box and/or to transmit that data to a receiver. In addition, in some embodiments, the lights, fan, or other components of the light box can be controlled remotely, e.g., so that the light cycle can be altered or so that the fan speed can be changed as necessary based upon exterior temperature changes. Thus, in some embodiments, the microprocessor is configured to control the speed of the fan and/or the intensity of the one or more light sources to adjust conditions inside the interior space of the main body.

In some embodiments, the light box can include a watering system, e.g., to drip and/or spray water onto plants grown in pots housed within the light box. The watering system can be programmed and controlled (e.g., via the microprocessor).

In some embodiments, the light box comprises at least one inlet for introducing a gas into the interior space. Thus, the inlet can be configured for attachment to a gas tank, canister, or other gas source. The gas tank, canister, or other gas source can provide a gas of interest, e.g., dry air, air of a predetermined or constant humidity, oxygen, carbon dioxide, nitrogen, argon, or any mixture thereof. Thus, in some embodiments, the use of the exhaust fan in combination with introduction of gas from the inlet can provide gas exchange, such that the presently disclosed light box can be used for studies related to anaerobic (hypoxic) conditions, stress with argon or nitrogen, or the effects of ethylene and/or CO₂. In some embodiments, the light box can be used to study plant growth under conditions that might be encountered during space travel.

In some embodiments, the light box further comprises one or more cameras (e.g., a Wi-Fi camera) configured to obtain images of the plants or microorganisms inside the interior space, e.g., to monitor growth without the need to open the box. In some embodiments, the camera can be configured to not only capture images from the interior space but also to transmit and/or record the images. In some embodiments, the camera is mounted on a vertical or horizontal track attached to one of the top, left, right, or rear side of the main body. Thus, in some embodiments, the camera position can be adjusted vertically or horizontally (e.g., remotely via a wireless communications device which can optionally be configured for communication with a microprocessor and/or motor configured to move the camera along the track). In some embodiments, the camera can be used to provide time lapse images of plant or culture growth.

Power for the components of the light box can be from any suitable source. In some embodiments, the light box further comprises a battery or a power cord.

Accordingly, it is an object of the presently disclosed subject matter to provide a light box for maintaining biological matter. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description and figures.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (sometimes schematically). A further understanding of the presently disclosed subject matter can be obtained by reference to an embodiment set forth in the illustrations of the accompanying drawings. Although the illustrated embodiments are merely examples of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

FIG. 1A is a schematic side view of an example embodiment of an enclosure for growing biological matter therein.

FIG. 1B is a top internal view of the enclosure of FIG. 1A.

FIG. 2A is an external isometric view of the enclosure of FIG. 1A.

FIG. 2B is an internal view of the top section of the enclosure of FIG. 2A.

FIG. 2C is an isometric view of the enclosure of FIG. 2A, in which the access door is open and the top section is removed to show a portion of the internal space of the enclosure.

FIG. 3 is an example embodiment of a fluid flow path between a condenser located within the base section of the enclosure of FIG. 2A and a reservoir containing a fluid with a pump for controlling the temperature within the enclosure.

FIG. 4 is an internal view of the enclosure of FIG. 2A, with biological specimens contained therein.

FIG. 5 is an isometric view of a light controller for the enclosure of FIG. 2A.

FIGS. 6A and 6B are respective isometric views of a controller and imaging device, the imaging device being movably mounted on a rail that is configured for mounting within the enclosure of FIG. 2A to record images and/or videos inside the enclosure without having to open the enclosure.

FIG. 7 shows example images within the enclosure recorded by the imaging device of FIGS. 6A and 6B, in which the images were illuminated with red light, white light, or blue light.

FIG. 8 is a rear isometric view of the enclosure of FIG. 2A.

FIG. 9 is an internal view of the enclosure of FIG. 2A, showing the vertically mobile lighting system installed therein.

FIG. 10A is an isometric view of a top section for use with an enclosure according to a second example embodiment.

FIG. 10B is an isometric view of an enclosure according to the second example embodiment, suitable for use with the top section shown in FIG. 10A.

FIG. 11 is an example embodiment of a vertical expansion unit for use in an enclosure disclosed herein.

### DETAILED DESCRIPTION

The terminology used herein is for the purpose of describing particular embodiments only and is not intended to limit the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a light source” refers to one or more light sources, including a plurality of the same type of light source. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of temperature, time, concentration, length, width, height, and so forth used in the specification and claims are to be understood as being modified in all instances by the term “about”. The term “about”, as used herein when referring to a measurable value such as an amount of mass, weight, time, volume, length, width, or temperature is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed subject matter. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms.

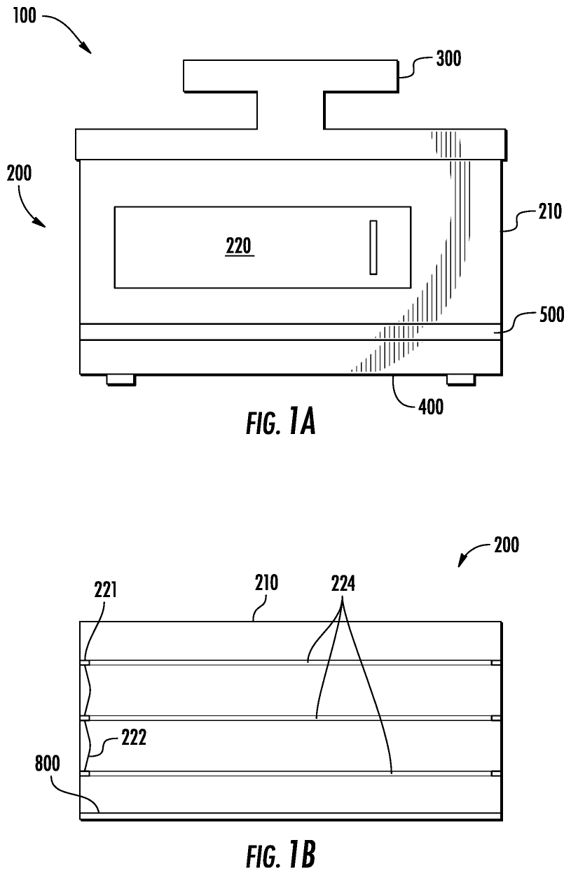

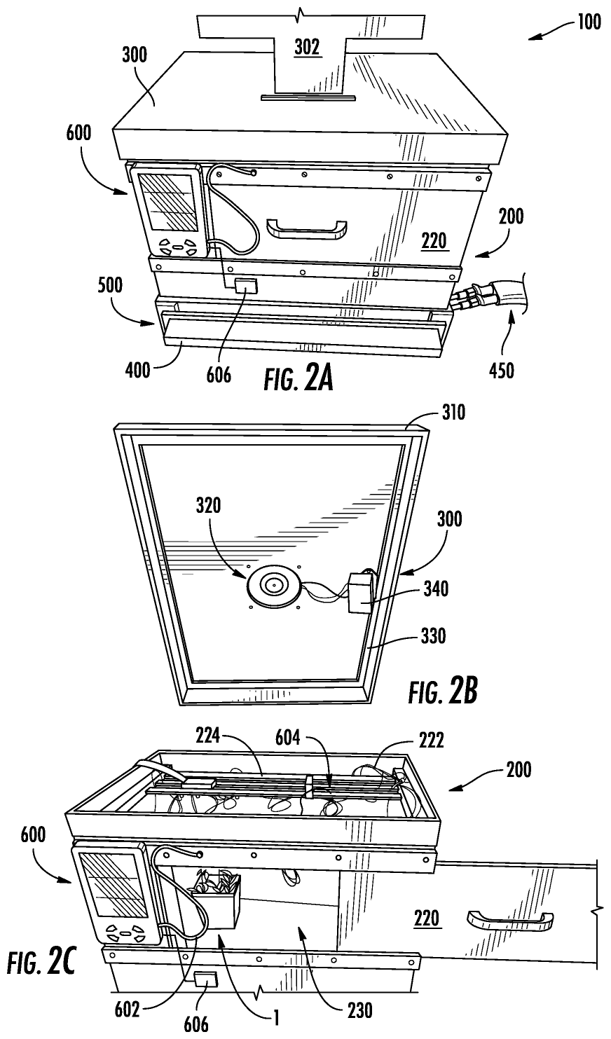

FIG. 1A shows a side view of a first example embodiment of an enclosure, generally designated 100, which can also be referred to as a light box and/or growth chamber interchangeably. The enclosure comprises a main body, generally designated 200, a removable top section 300, optional air inlet vents 500; and a base 400. As shown, the main body 200 includes a box 210, which has a door 220 covering an opening (see, e.g., opening 230, FIG. 2C) formed in the box 210. In the example embodiment shown, the door 220 is a sliding door, however any suitable door (e.g., a pivoting door, a segmented door, and the like) can be used without deviating from the scope of the subject matter disclosed herein. The top section 300 is removably and, optionally, self-sealing when positioned over the main body 200. The top section 300 includes an exhaust fan and outlet vents, such that air within the main body 200 can be exhausted therefrom through the top section 300, with fresh air being drawn into the enclosure 100 through the air inlet vents 500 to replenish the air supply within the enclosure 100.

FIG. 1B is a top view of the main body, generally designated 200, of the enclosure (e.g., 100, FIG. 1A), in which the top section (e.g., 300, FIG. 1A) is removed to show an interior of the main body 200 and, specifically, of the box 210. The box 210 has a plurality of vertically-extending rails 221 attached to the internal surface thereof. The rails 221 extend to have a length that is all or a portion of the height of the interior of the box 210. The rails 221 are arranged in pairs on opposing sides of the box 210. A plurality of light emitter panels 224 are provided, each light emitter panel 224 being mounted on and extending between an opposing pair of the rails 221. The rails 221 have a plurality of positions along the length thereof at which the light emitter panels 224 can be attached within the box 210. As such, each of the light emitter panels 224 can be mounted between a corresponding pair of rails 221 at one of a plurality of heights within the box 210.

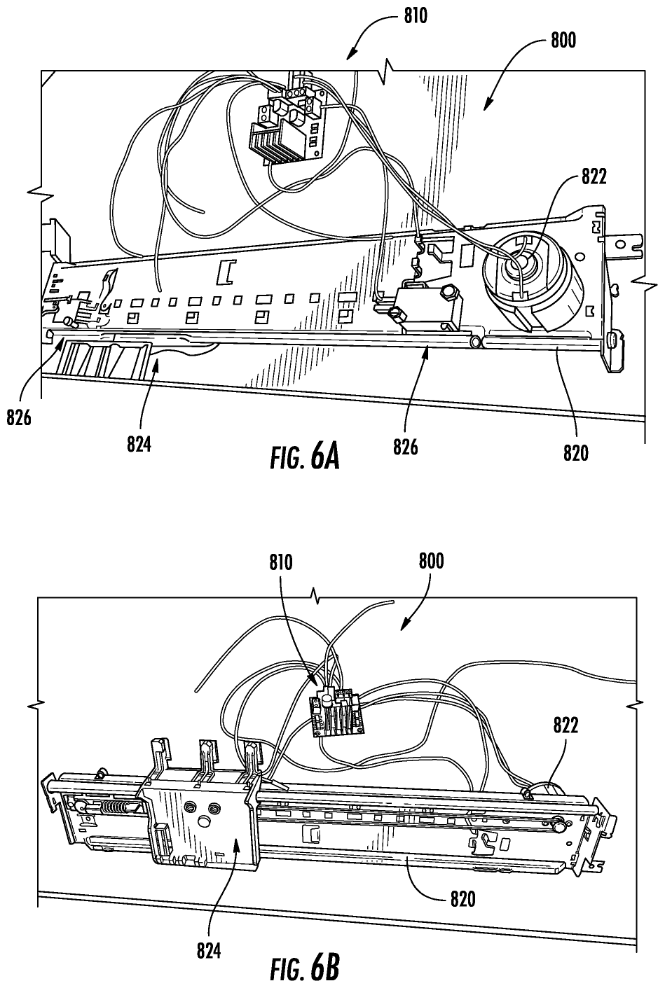

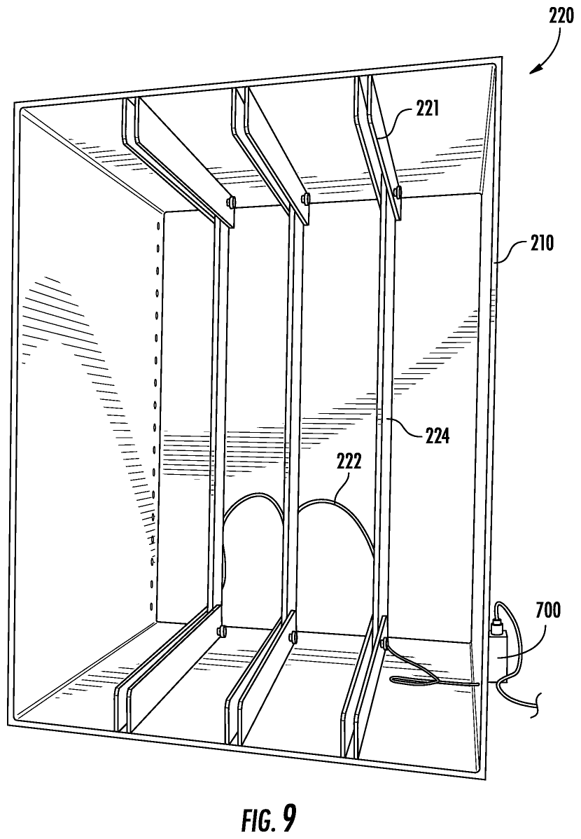

In some embodiments, each light emitting panel 224 can be positioned at the same height within the box 210. In some embodiments, each light emitting panel 224 can be positioned at a height within the box 210 independent of others of the light emitter panels 224. This can be advantageous to provide appropriate levels of illumination within the box 210 when biological matter (e.g., plants) of different heights are positioned within the box 210. While any suitable light source may be used for the light emitting panel(s) 224, strings of light emitting diodes (LEDs) are used in the example embodiment disclosed herein. As such, in the example embodiment disclosed herein, the LEDs are attached (e.g., using an adhesive) to a suitably rigid bracket, the bracket comprising members (e.g., a hook) configured to be received within a receiver (e.g., a hole or rod) formed in the rails 221. The light emitter panels 224 are electrically connected together by a wire 222 (also shown in FIG. 9). In some embodiments, the light emitter panels 224 are electrically connected independent of each other, so that a light output intensity and/or color of each light emitter panel 224 can be the same or different from others of the light emitter panels 224. In the example embodiment shown, the box 210 comprises a movable imaging system 800, which will be described further elsewhere herein regarding FIGS. 6A and 6B.

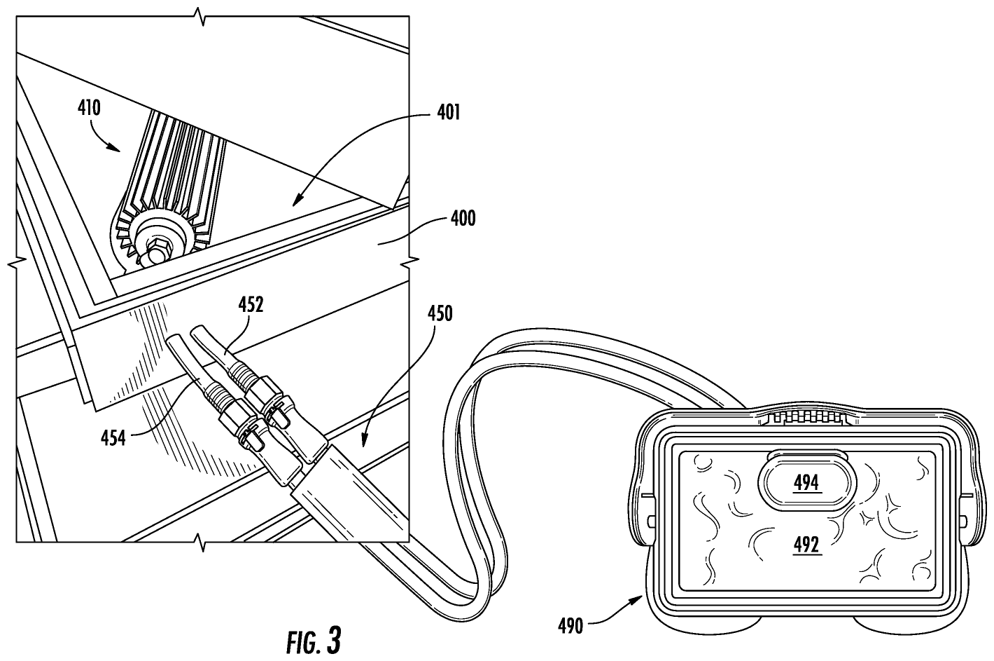

FIG. 2A is an isometric front view of the enclosure 100, in which an environmental monitoring system, generally designated 600, is attached to an outer surface of the main body 200 to display, for example, temperature and humidity data from within the enclosure 100 and, specifically, of the environmental conditions prevalent within the main body 200. As such, the environmental monitoring system 600 is connected to temperature and/or humidity sensors positioned within the main body 200. The main body 200 comprises a sliding front access door 220 to provide access to the interior space of the main body 200. The enclosure 100 comprises a base 400 with an interior that can be accessed by lifting the main body 200 off of the base 400 (e.g., so that a condenser inside the base can be accessed). A water supply connector 450 is provided into the base 400 to supply a fluid (e.g., water) at a specified temperature to maintain a predetermined temperature within the main body 200 of the enclosure 100. The water supply connector 450 is connected to a condenser (see 410 in FIG. 3) in the base 400.

As shown in FIG. 2B, the enclosure 100 comprises a removable top section 300 (e.g., a lid) which, when removed, provides access to the interior space of the main body 200 of the enclosure 100 from the top (e.g., from the direction shown in FIG. 1B). The top section 300 has a vertically-extending T-shaped member 302, which houses an exhaust fan, generally designated 320, and vents for exhausting air flowing through the exhaust fan 320 to the external environment of the enclosure 100. The top section 300 advantageously comprises a slot and/or gasket 330, which seals against the upper surface of the main body 200 to prevent air flow from being drawn into the enclosure 100 without passing through the base 400 (e.g., adjacent to the condenser therein). The top section 300 has a fan controller 340 attached thereto, which is electrically connected to the exhaust fan 320 and controls a speed of the exhaust fan 320 and an air flow rate through the exhaust fan 320 and correspondingly, an air flow rate through the enclosure 100.

In FIG. 2C, the door 220 is shown in an open position, such that an interior of the main body 200 is accessible through the open door 220, as well as through the top thereof, when the top section 300 is removed, as shown in FIG. 2C. As such, the organic matter, generally designated 1, is visible and/or accessible by opening and/or removing the top section 300 and/or through the opening, generally designated 230, when the opening 230 is not covered by the door 220. The door 220 can take the form of any suitable cover. As shown, the light emitter panels 224 are positioned at a top of the main body 200 and the environmental monitoring system 600 has a wire 602 in electrical communication with environmental sensor(s), generally designated 604, positioned and/or arranged within the main body 200.

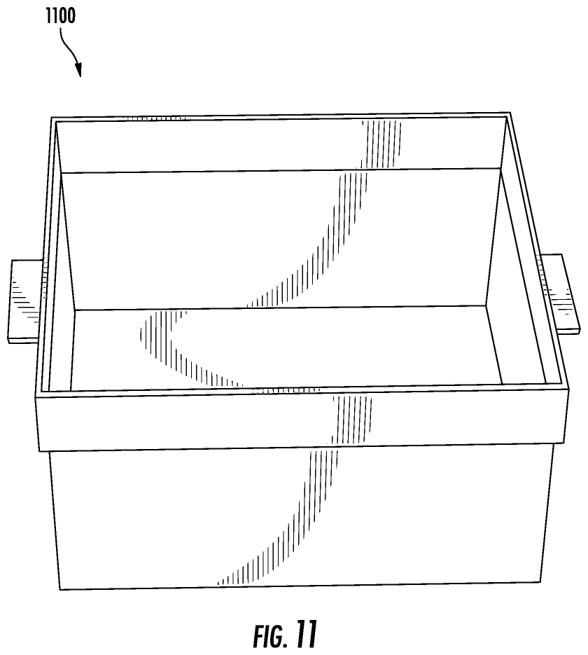

In the example embodiment of the enclosure shown in FIGS. 2A-2C, the enclosure 100 has the following dimensions: length of 19 inches, width of 14 5/32 inches, and height 18⅛ inches; with the top section having a height of 6¼ inches. In some embodiments, the enclosure 100 can be powered by 12 Volts AC or DC. In some embodiments, the enclosure is configured to receive power, either entirely from or partially from (e.g., for a specific period of time, such as during transport of the enclosure 100) a portable power source, such as an internal and/or external battery. The main body 200 of the enclosure 100 can be replaced, if desired, with lighter and/or more transparent material depending upon the particular application for which the enclosure 100 is to be used. Alternatively, an extendable main body 200 can be provided (e.g., having metal extenders). An example of such an expandable portion, generally designated 1100, of the main body 200 is shown in FIG. 11. The main body 200 can include one or more inlets for receiving a gas (e.g., having a composition different from the composition of ambient air surrounding the enclosure 100). Thus, the enclosure 100 can be attached to a gas canister, gas tank, or any other suitable gas source for studies where the effects of particular atmospheric conditions on the growth and/or reproduction of biological matter within the enclosure are to be studied. The main body 200 can also be fitted with a programmable irrigation system (e.g., a water dripping system) of the type that is suitable for growing potted plants inside the enclosure 100. In addition to the environmental monitoring system 600 shown in FIGS. 2A and 2C and the environmental sensor(s) to which it is electrically connected, the enclosure can also include additional sensors (e.g., light intensity sensors and/or soil moisture sensors), which can be integrated within the main body 200. In some embodiments, the one or more sensors can be mounted in an interior of the of the enclosure 100 for detecting and/or monitoring environmental conditions within the enclosure 100 (e.g., within the main body 200). In some embodiments, the one or more sensors include an external sensor 606 mounted on an exterior of the enclosure (e.g., on the main body 200 and/or at and/or adjacent to the air inlet vents 500) for detecting and/or monitoring environmental conditions (e.g., air temperature, air humidity, air composition, and the like) external to the enclosure 100. In some embodiments, such an external sensor 606 may be provided at the air inlet vents 500 to detect environmental parameters of an air stream drawn into the enclosure 100 from the exterior of the enclosure 100. An example embodiment of such an external sensor 606 is shown in FIG. 2A, however the position shown in FIG. 2A for the placement of the external sensor 606 is not limited to that which is shown in FIG. 2A.

FIG. 3 shows an example embodiment of a condenser, generally designated 410, that can fit into a cavity, generally designated 401, formed in the base 400 of the presently disclosed enclosure 100 and a reservoir, generally designated 490, containing a fluid 492 at a preferred temperature (e.g., an ice/cold water reservoir, sometimes referred to as an Iceman reservoir) for maintaining a desired temperature within the enclosure 100. The reservoir 490 has a pump 494 that can be used to pump the fluid 492 from within the reservoir 490 into the condenser 410 via a fluid inlet line 452, which is removably coupled to the reservoir 490 at the fluid coupler, generally designated 450. A fluid return line 454 is also provided at the fluid coupler 450 to allow the fluid 492 to flow, after flowing through and chilling the condenser 410, back into the reservoir 490. In some embodiments, in addition to or instead of the reservoir 490, the fluid inlet line 452 can be attached to a substantially continuous and/or limitless supply of fluid (e.g., a faucet that is capable of providing a continuous flow of water, and preferably a continuous flow of cold or chilled water).

In some embodiments, in which the light emitter panels 224 are set to emit the highest light intensities, and operating the exhaust fan 320 at a maximum fan speed, a reservoir 490 as shown herein filled initially with ice and water can maintain a temperature within the main body 200 of the enclosure 100 at about 14° C. However, in embodiments in which the condenser 410 is omitted, the temperature within the main body 200 can rise to at least about 28-30° C. or more, unless the box is kept in cool ambient temperatures (e.g., by drawing in sufficiently cool air through the air inlet vents 500 and exhausting air from within the main body 200 through the exhaust vents of the top section 300, using the exhaust fan 320).

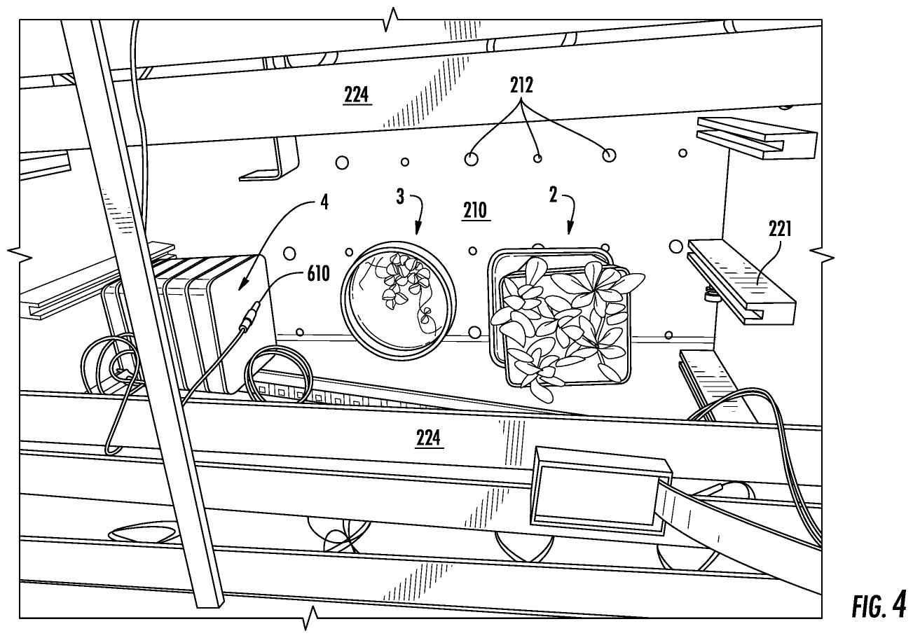

FIG. 4 is a top view of the interior space of within the main body 200 of the enclosure 100, such as is shown in FIG. 2A. The bottom surface of the main body 200 (e.g., the surface adjacent to the base 400) has a plurality of holes 212 formed through an entire thickness thereof. The holes 212 are configured to provide a flow of chilled air from the cavity 401 of the base 400 after having been chilled by the condenser 410 in the base 400. As shown, the enclosure 100 is suitable for monitoring the growth of: biological matter, including potted plants 2, seedlings growing in a round plate 3, and/or seedlings growing in vertical plates 4. Light emitter panels 224 comprise a plurality of LEDs attached on an aluminum base plate extending between opposing rails 221, the light emitter panels 224 being used to illuminate the interior of the main body 200.

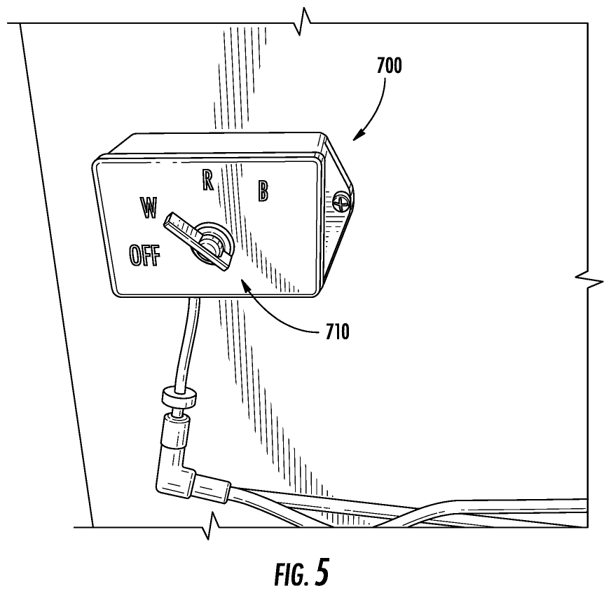

FIG. 5 shows an example embodiment of a light controller, generally designated 700, mounted on the exterior of the main body 200 of the enclosure 100. The light controller 700 is configured to control the type (e.g., the color and/or intensity) of light illuminating the interior of the main body 200 (e.g., by the light emitter panels 224). The light controller 700 shown in the example embodiment of FIG. 5 can be used to allow via switch 710 a user to choose any of turning the light emitter panels 224 off or to emit any of white light W, far red light R (e.g., having a peak wavelength of between 720 nm and 740 nm), and/or blue light B (e.g., having a peak wavelength between 465 nm and 485 nm). In some embodiments, only one type (e.g., white or far-red or blue light) or any combination of light (e.g., far-red with blue) can be used to illuminate the interior of the box. While there are three colors of light shown as being user-selectable for the light controller 700, any suitable number of different colors, including colors other than white, red, and blue, may be included without deviating from the scope of the subject matter disclosed herein.

FIGS. 6A and 6B show front and back views of a camera mount slider system, generally designated 800, suitable for use in an enclosure 100 according to the presently disclosed subject matter. FIG. 6A (e.g., the front view) shows a stepper motor 822 and reset switches 826 for the camera mount, while FIG. 6B (e.g., the back view) shows the camera mount slider 824. The movement of the camera mount slider 824 is defined by the length of the rail 820 and is controlled by a controller, generally designated 810, which is in electronic communication with the stepper motor 822, the reset switches 826, and the camera mount slider 824. The distance between the reset switches 826 defines the length of travel permitted for the camera mount slider 824 along the rail 820.

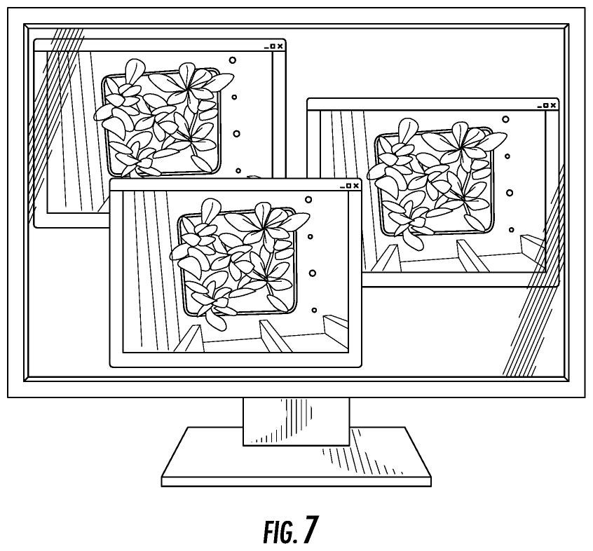

FIG. 7 schematically shows a computer screen displaying photos of plants grown in a light box of the presently disclosed subject matter under illumination with different light colors (e.g., blue, far red, and/or white) captured and transmitted from a controller (e.g., 810, such as a Raspberry Pi) for a camera arranged inside the enclosure 100.

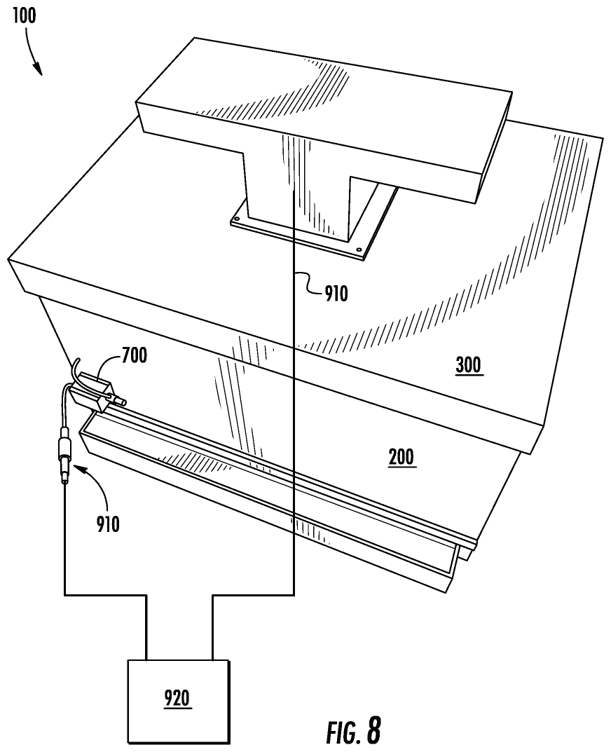

FIG. 8 is an isometric rear view the example embodiment of the enclosure 100 of the presently disclosed subject matter. The enclosure 100 can have a black or white exterior color. The top section 300 (e.g., the lid) of the enclosure 100 can include an exhaust fan (e.g., in a T-shaped structure on the lid) and can be removable. The interior of the main body 200 and/or of the top section 300 is preferably white for high light reflection. Illumination of the interior of the main body 200 can be achieved with light emitter panels 224 (better seen in FIG. 1B) that can be height, color, and/or intensity adjusted. Inlet vents 500 are designed to obstruct external light from entering the interior of the main body 200 while providing air flow into and/or through the main body 200 of the enclosure 100. The example embodiment of the enclosure 100 is about 19 inches long, about 14 5/32 inches wide, and about 12⅛ inches high (i.e., with the top section 300 removed). The top section 300 with the exhaust fan has a height of about 6¼ inches. FIG. 8 shows a power supply 920 connected to the top section 300 and to the light controller 700 (also shown in FIG. 5) by wires 910. As such, the top section 300 and the light controller 700 can be independently controlled.

FIG. 9 shows a top view of the interior space of the main body 200 of the enclosure 100, in which three light emitter panels 224 are positioned at different positions along the width of the main body 200, suspended between in rails 221 attached to the inside of the sides of the main body 200 (e.g., the inside walls). The light emitter panels 224 can be secured at any of a plurality of heights within the main body 200, so as to accommodate biological matter having different heights therein. In some embodiments, some of the light emitter panels 224 may be positioned at different heights from others of the light emitter panels 224.

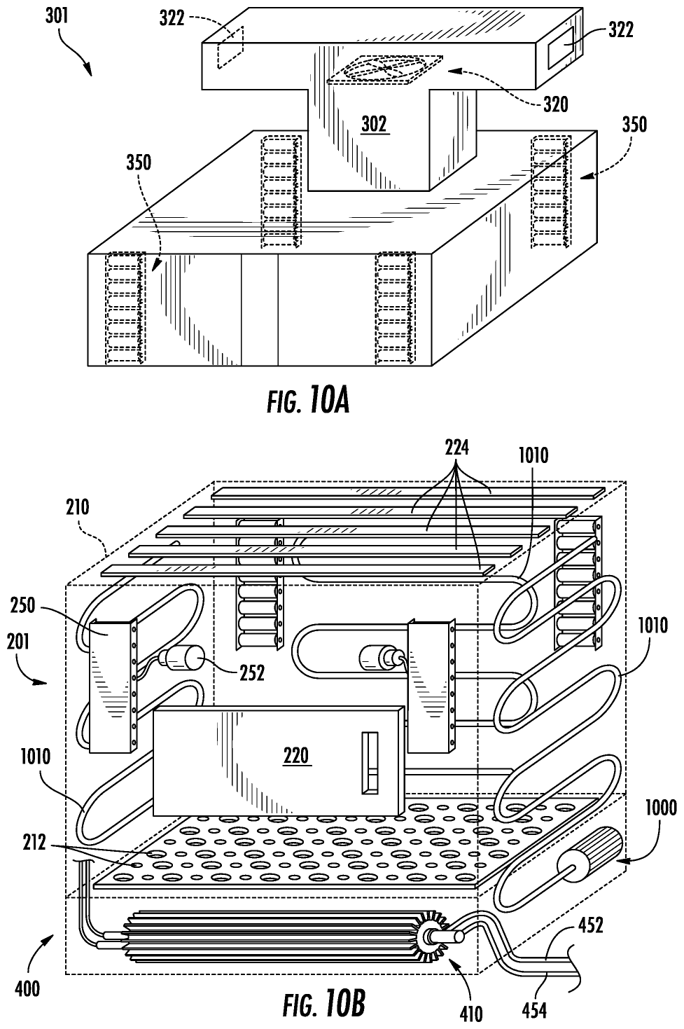

FIGS. 10A and 10B show various aspects of a second example embodiment for an enclosure (e.g., a “light box”) according to the subject matter disclosed herein. The enclosure comprises, as shown in FIG. 10A, a top section, generally designated 301, which includes a generally T-shaped portion 302, in which an exhaust fan 320 and exhaust ports 322 are provided. The exhaust fan 320 is arranged so as to blow air out of the exhaust ports 322 when the exhaust fan 320 is operational (e.g., is spinning). The placement of the exhaust fan 320 and the exhaust ports 322 are merely exemplary and the exhaust fan 320 and the exhaust ports 322 may be positioned within the top section 301 at any position suitable for exhausting air from the main body, generally designated 201 in FIG. 10B.

FIG. 10B shows further aspects of the second example embodiment of the enclosure, specifically of a main body 201 that is attached to a base, generally designated 400. The exterior walls of the main body 201 and the base 400 are illustrated schematically in broken line so as to better illustrate the internal features contained within the main body 201 and the base 400. The main body 201 comprises a door 220 that covers, when closed, an opening (see, e.g., opening 230, FIG. 2C) in a side of the main body 201, to allow to access within the main body 201 without having to remove the top section 301 from the main body 201. One or more of the side walls of the main body 201 advantageously have a dual-layer construction, such that an external wall of the main body 201 is visible from the exterior of the main body 201 but not from the interior of the main body 201 and an internal wall of the main body 201 is visible from the interior of the main body 201 by not from the exterior of the main body 201. The interior and exterior walls of the main body 201 are advantageously separated from each other by an air gap.

In the example embodiment shown, the gap between the interior and exterior walls of the main body 201 has a thermoelectric coil 1010 arranged therein to control a temperature within the interior of the main body 201. The thermoelectric coil(s) 1010 are operatively connected to a thermoelectric device, generally designated 1000, which generates a temperature gradient using an electrical current from a power source (e.g., a battery or any other suitable power source). The thermoelectric coil(s) 1010 may have any suitable number of turns and may occupy any suitable percentage of the height and/or width of the side walls of the main body 201.

The base 400 has a condenser, generally designated 410, which is connected to a fluid inlet line 452 to convectively cool or heat air surrounding the condenser 410 when a heated or chilled fluid is passed therethrough. The base 400 can also have a fluid return line 454, which recirculates the fluid back into the reservoir (e.g., 490, FIG. 3), or otherwise to a drain, after the fluid has passed through the condenser 410. The base 400 has air inlets formed therein (e.g., in a bottom or side(s) thereof) to allow for ambient air (e.g., air external to the base 400 and the main body 201) to be drawn into the base 400 and conditioned (e.g., heated or cooled, based on whether the fluid flowing through the inlet fluid pipe 452 is warmer or cooler than the ambient air), after which the air is drawn (e.g., by suction, due to operation of the exhaust fan 320) through the holes 212 formed in the bottom surface of the main body 201, where the biological matter is located within the main body 201.

As such, the enclosure of the second example embodiment comprises two types of cooling. The first cooling type is evaporative cooling using the condenser 410, which advantageously can help in accumulating humidity within the interior of the main body 201. The second cooling type is thermoelectric using the thermoelectric device 1000 and the thermoelectric coil(s) 1010, which are capable of providing additional precision in temperature control and allow for maintaining any of a plurality of temperatures over a range of temperatures within the interior of the main body 201.

The top section 301 has a plurality of tracks, generally designated 350, which are rigidly connected to the top section 301. The tracks 350 may be attached on an external surface of the top section 301 or an internal surface of the top section 301. The main body 201 has tracks 250, which are complementarily shaped to the tracks 350 of the top section 301. The main body comprises a high torque, low RPM motor 252 that causes an activation of the tracks 250, such that a vertical position of the top section 301 relative to the main body 201 is adjustable. As such, an effective vertical height of the interior space within the main body 201 can be controlled by controlling a position of (e.g., by lowering and/or raising) the top section 301 relative to the main body 201. The enclosure comprises a plurality of light emitter panels 224, which are advantageously rigidly attached to the top section 301, for illuminating the interior of the main body 201 at a prescribed luminous intensity.

Such vertical adjustability of the top section 301 relative to the main body 201 is advantageous because the height at which the light emitter panels 224 attached to the top section 301 can be controlled based on a vertical height of the biological matter (e.g., plants) contained within the main body 201. In some embodiments, the vertical position of the top section 301 relative to the main body 201 is controlled manually by a user (e.g., by monitoring a change in vertical height of plants, for example, contained within the main body 201. In some embodiments, the main body 201 and/or the top section 301 comprises a proximity sensor, or other suitable distance measuring device, and can detect a change in distance between the biological matter contained within the main body 201 and the top section 201 upon vertical growth of the biological matter and can automatically engage the motor(s) 252 to cause a relative vertical movement between the top section 301 and the main body 201 (e.g., between tracks 250, 350) to automatically maintain a predetermined distance (e.g., correlated to a desired luminous intensity) between the light emitter panels 224 of the top section 301 and the biological matter within the main body 201.

In the embodiments shown, the tracks 250 and the tracks 350 are attached to the main body 201 and the top section 301, respectively, at positions so that each track 250 is positioned to engage with a corresponding track 350 when the top section 301 is installed over the main body 201. The tracks 250, 350 may be arranged in any suitable pattern, but are shown in the example embodiment as being arranged such that pairs of tracks 250, 350 are arranged on opposing sides of the enclosure. In some embodiments, the tracks 250 and/or 350 may take the form of gears with a plurality of teeth and/or a chain and sprocket arrangement. Either of the tracks 250 or 350 may be provided with and driven by the motors 252. In some embodiments, a single motor 252 can be provided to control the movement of a plurality of tracks 250 or 350. Regardless of the number of motors 252 provided, the motors 252 are connected to a controller so as to operate synchronously to ensure that the top section 301 does not become inclined or otherwise misaligned relative to the main body 201, which can lead to a malfunction (e.g., a “jam”). A pulley system can be provided to connect the motor(s) 252 to the tracks 250 or 350. For example, as the height of the biological matter (e.g., plants) within the main body 201 increases, a proximity sensor in the top section 301 is configured to send a signal to an electronic controller in the base 400, which activates the motors 252 to rotate their respective tracks 250. The clockwise and counter-clockwise rotation of the tracks 250 will result in the vertical lift of the top section 301 relative to the main body 201.

In some embodiments, slots may be provided on the side(s) of the external surface of the main body 201 and to the top section 301 to add two or more main bodies 201 over a single base 400. In some embodiments, the top section 300 of all main bodies 201 other than the top-most main body 201 in the stack may be modified to not have the T-shaped handle portion and to only be a perforated sheet (e.g., with holes, such as holes 212) to allow for air to pass between adjacent main bodies 201, and to have light emitter panels 224 attached thereto to allow for illumination of each main body 201 independent of each other main body 201.

In some embodiments, in order to allow for relative movement between the top section 301 and the main body 201, the top section 301 is either dimensionally (e.g., cross-sectionally) larger or smaller than the main body 201 so that the top section 301 can be fitted within or around the main body 201 (e.g., in a nested configuration). As such, in order to maintain adequate control of the environmental conditions within the main body 201, it is advantageous to provide a sealing member (e.g., a gasket or flexible flange) on the surface of the top section 301 on which the tracks 350 are provided and/or on the surface of the main body 201 on which the tracks 250 are provided, so as to prevent the infiltration of ambient light and/or ambient air into the main body 201 at the region where the top section 301 is movably joined to the main body 201. As such, intrusion of ambient light and/or ambient air into the main body 201 is prevented by the sealing member even when the top section 301 is moving relative to the main body 201.

The presently disclosed light box provides several advantages. Due to its relative small size and light weight compared to commercial growth boxes, the presently disclosed light box is portable, making it possible to transport the light box to any desirable location, e.g., near a field where the biological matter is to be grown. The combination of light weight and low cost can further facilitate the use of the light box in a variety of settings, e.g., in schools, on farms, or in the context of the transportation of biological matter (e.g., an organism) that require particular conditions. In some embodiments, the light box can be used to transport plant organisms used as food (e.g., vegetable greens, such as lettuces, spinach, and microgreens; herbs, berries, etc.), so that the food plants can be grown during transport, avoiding spoilage in plant foods having a short shelf life and/or providing such food plants to regions not amenable to the growth of the food plants or when the food plant is out of season. Accordingly, in some embodiments, the presently disclosed subject matter provides a method of transporting a food item, wherein the method comprises maintaining (which can comprise growing) the food item in a light box of the presently disclosed subject matter while simultaneously transporting the light box to a desired location.

The growth of the food plants in the light box can also protect the plant from organisms (e.g., Salmonella or other pathogens) that can cause disease to humans or other animals that consume the food plants or that can harm the plants themselves. For example, the boxes can protect the plant foods from insect pests as well as microorganisms. Because of the relatively small size of the presently disclosed boxes, groups of the boxes can be readily used to test multiple growth conditions and/or to grow multiple different types of plants in the same amount of space without the use of multiple greenhouses.

In some embodiments, the presently disclosed light box can be used to modulate the growth rate of a plant or microorganism. For instance, the light box can be used to increase the rate of growth or decrease the rate of growth of a plant grown inside the box by adjusting one or more conditions such as, but not limited to, light, temperature, humidity, atmosphere, gas, etc. Thus, in some embodiments, the presently disclosed subject matter provides a method of adjusting the growing cycle of a plant by adjusting conditions within the box in which the plant is grown to maintain, increase, and/or slow down the plant's growth. This method can provide the delivery of plant foods to a desired location at a desired point in the plant's growing cycle, e.g., in cases where longer transport distances would otherwise result in the delivery of a plant food after the plant had started to spoil. This method can also be used to extend the growing season of a food item even in the absence of using the light box for transport, e.g., to provide otherwise out of season food items more generally.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. An enclosure for maintaining biological matter therein under controlled conditions, the enclosure comprising:
a main body comprising an interior space accessible from an exterior of the enclosure;
one or more light emitter panels configured to illuminate the interior space;
a base configured to receive the main body thereon;
a cooling system located within the base; and
wherein the enclosure is configured to maintain a temperature within the interior space between about 14 degrees Celsius (° C.) and about 30° C.

2. The enclosure of claim 1, wherein the main body comprises an opening with a movable door, through which the interior space is accessible and/or wherein the system comprises a top section that is removably attached to a top surface of the main body.

3. The enclosure of claim 2, wherein the main body has a substantially cuboid-shaped structure comprising a bottom side, a front side, a rear side, a left side, and a right side which define the interior space therein, wherein the door is formed in the front side.

4. The enclosure of claim 3, wherein the top section comprises an exhaust fan and/or wherein the base comprises one or more air inlet vents, the exhaust fan being configured to induce an airflow into the base through the one or more air inlet vents and to exhaust air from the main body through the top section.

5. The enclosure of claim 4, wherein the bottom side of the main body comprises one or more holes through a thickness thereof, wherein the exhaust fan is configured to induce an air flow from the base into the main body through the one or more holes for cooling of the interior space.

6. The enclosure of claim 4, wherein the one or more light emitter panels are positioned near the exhaust fan for removing heat from the interior space by the exhaust fan.

7. The enclosure of claim 1, wherein the biological matter comprises photosynthetic organisms, food material, and/or bacteria and/or yeast cultures.

8. The enclosure of claim 1, wherein the cooling system comprises a container, a fluid inlet for introducing a fluid into the container, and a fluid outlet for removing the fluid from the container.

9. The enclosure of claim 8, comprising a reservoir and a pump, which is configured to pump the fluid from the reservoir into the fluid inlet, wherein the reservoir contains water or water and ice.

10. The enclosure of claim 8, wherein the container is a metal coil or a metal cylinder comprising one or more projections to increase the surface area of the container.

11. The enclosure of claim 10, wherein the metal coil or the metal cylinder is made of stainless steel and/or Heresite P413 or E-coated aluminum and/or copper nickel alloy.

12. The enclosure of claim 1, wherein the one or more light emitter panels comprise light emitting diodes (LEDs) rigidly attached to brackets suspended over the interior space of the main body to illuminate biological matter contained therein.

13. The enclosure of claim 12, wherein the LEDs are configured to emit one or more of white light, far red light, and/or blue light.

14. The enclosure of claim 1, comprising one or more sensors arranged within the interior space to measure one or more environmental conditions inside the interior space and/or arranged on an exterior of the enclosure for detecting and/or monitoring environmental conditions external to the enclosure.

15. The enclosure of claim 14, wherein the one or more sensors comprise one or more of a light intensity sensor, a soil moisture sensor, a temperature sensor, an oxygen sensor, a carbon dioxide sensor, and a humidity sensor.

16. The enclosure of claim 15, comprising an environmental monitoring system comprising a microprocessor in electronic communication with the one or more sensors, wherein the environmental monitoring system is configured to show on a display one or more environmental conditions and/or to transmit data related to the one or more environmental conditions to a receiver.

17. The enclosure of claim 16, wherein the receiver is a component of a personal computer or personal electronic device, so that the one or more environmental conditions can be monitored remotely from the enclosure.

18. The enclosure of claim 16, comprising an exhaust fan for providing an air flow through the enclosure, wherein the microprocessor is configured to control a speed of the exhaust fan and/or an intensity of the one or more light emitter panels to adjust one or more environmental conditions within the interior space.

19. The enclosure of claim 18, comprising a programmable irrigation system in a form of a drip system and/or a spray system to supply water to the biological matter within the interior space of the main body.

20. The enclosure of claim 19, wherein the microprocessor is configured to control the programmable irrigation system in response to the one or more environmental conditions detected by the one or more sensors within the interior space of the main body.

21. The enclosure of claim 1, comprising a gas inlet, by which a gas is configured to be introduced into the interior space.

22. The enclosure of claim 21, wherein the gas comprises one or more of dry air, air of a predetermined humidity, oxygen, carbon dioxide, nitrogen, argon, or a mixture of thereof.

23. The enclosure of claim 1, comprising a camera configured to capture, transmit, and/or store one or more images from the interior space.

24. The enclosure of claim 23, wherein the camera is mounted on a track attached to an interior surface of the main body, such that a position of the camera within the interior space can be adjusted to view different regions within the interior space.

25. The enclosure of claim 24, wherein the position of the camera can be adjusted remotely via a wireless communication device.

26. The enclosure of claim 25, wherein the wireless communication device is configured for communication with a microprocessor.

27. The enclosure of claim 24, wherein the track is vertically oriented or horizontally oriented within the main body.

28. The enclosure of claim 1, comprising a battery or a power cord for supplying power to the one or more light emitter panels and/or to a fan configured to provide an air flow through the enclosure.

29. The enclosure of claim 1, wherein the main body is colored or coated to prevent ambient light from reaching the interior space.

30. The enclosure of claim 1, comprising a top section that is removably attached to a top surface of the main body, wherein the top section and the main body comprise a plurality of complementarily-shaped tracks, and wherein the top section or the main body comprise one or more motors configured to be activated to cause a relative movement between the top section and the main body to change a height of the interior space.

31. The enclosure of claim 30, wherein the one or more motors are a plurality of motors, and wherein the enclosure is configured to control the plurality of motors to be activated synchronously to ensure that the orientation between the top section and the main body does not change while the top section moves relative to the main body.

32. The enclosure of claim 30, wherein the one or more light emitter panels are attached to the top section, the top section comprising a sensor configured to detect a distance between the sensor and the biological matter contained within the interior space.

33. The enclosure of claim 32, wherein the top section is configured to move relative to the main body to alter a height of the interior space in response to a change in height of the biological matter contained within the interior space.

34. The enclosure of claim 1, comprising a first cooling system and a second cooling system;
wherein the first cooling system comprises a condenser within the base, the condenser being configured to receive a fluid therein to alter a temperature of an air flow through the base;
wherein one or more lateral walls of the main body comprises an internal wall and an external wall, the internal wall being spaced apart from the external wall by a gap;
wherein the second cooling system comprises a thermoelectric device operatively connected to one or more thermoelectric coils arranged between the internal wall and the external wall of one or more lateral walls of the main body.

35. A method of maintaining biological matter, the method comprising:
providing one or more enclosures, each enclosure comprising:
a main body comprising an interior space accessible from an exterior of the enclosure;
one or more light emitter panels configured to illuminate the interior space;
a base configured to receive the main body thereon, the base comprising a cooling system; and
wherein the enclosure is configured to maintain a temperature within the interior space between about 14 degrees Celsius (° C.) and about 30° C.; and
maintaining the biological matter in each enclosure.

36. The method of claim 34, wherein the main body of each enclosure comprises one or more of an opening with a movable door through which the interior space is accessible and/or a top section that is removably attached to a top surface of the main body.

37. The method of claim 35, further comprising transporting each enclosure to a desired location.

38. The method of claim 35, wherein the biological matter comprises one or more plant organisms and maintaining the one or more plant organisms comprises growing the one or more plant organisms.

39. The method of claim 38, comprising growing the one or more plant organisms while the one or more enclosures are transported to a desired location.

40. The method of claim 38, wherein growing the one or more plant organisms in the enclosure protects the one or more plant organisms from organisms that can cause disease to humans and/or other animals that consume the one or more plant organisms and/or that can harm the one or more plant organisms themselves.

41. The method of claim 40, wherein the organisms comprise insect pests and/or microorganisms.

42. The method of claim 38, comprising:
providing a plurality of enclosures; and
testing multiple growth conditions for the one or more plant organisms in each enclosure and/or growing different types of plant organisms in one or more of the enclosures.

43. The method of claim 38, comprising controlling one or more environmental conditions within each enclosure to modulate a growth rate of the one or more plant organisms within each enclosure.

44. The method of claim 43, wherein controlling one or more environmental conditions within each enclosure comprises increasing a growth rate of the plant organism or decreasing the growth rate of the one or more plant organisms within each enclosure.

45. The method of claim 44, wherein the one or more environmental conditions comprise light color, light intensity, temperature, humidity, atmospheric pressure, and/or atmospheric composition.

46. The method of claim 38, comprising adjusting one or more environmental conditions within the enclosure to maintain, slow, or increase a growth rate of the one or more plant organism, thereby adjusting a growing cycle of the one or more plant organisms

47. The method of claim 38, comprising delivering the one or more plant organisms to the desired location at a desired point in a growth cycle of the one or more plant organisms.
